(19) United States
(12) Patent Application Publication
(10) Pub. No.: US 2009/0049748 A1
(43) Pub. Date: Feb. 26, 2009

(54) METHOD AND SYSTEM FOR CONVERTING WASTE INTO ENERGY

(75) Inventors: Eric Day, Longmeadow, MA (US); Andrew Eric Day, Longmeadow, MA (US)

Correspondence Address: CANTOR COLBURN, LLP, 20 Church Street, 22nd Floor, Hartford, CT, 06103, US

(73) Assignee: Eric Day, Longmeadow, MA (US)

(21) Appl. No.: 12/201,558

(22) Filed: Aug. 29, 2008

Related U.S. Application Data
(63) Continuation-in-part of application No. 11/680,704, filed on Mar. 1, 2007.
Application No. 11/680,704 is a continuation of application No. 11/627,403, filed on Jan. 26, 2007.
Application No. 11/627,403 is a continuation of application No. 11/621,801, filed on Jan. 10, 2007.
Application No. 11/621,801 is a continuation of application No. 11/620,018, filed on Jan. 4, 2007.

(51) Int. Cl. C10J 3/68; C12M 1/00

(52) U.S. Cl. 48/77; 48/211; 435/289.1

(57) ABSTRACT

Disclosed herein is system comprising a system comprising a high temperature gasifier; the high temperature gasifier being operative to function at temperatures of greater than or equal to about 450° F.; and a synthesizing reactor in fluid communication with the high temperature gasifier; the synthesizing reactor being operative to convert carbon dioxide into another form of carbon. Disclosed herein too a method comprising gasifying a waste stream to produce a composition that comprises syngas; the syngas comprising carbon monoxide and hydrogen; the gasifying being conducted in a high temperature gasifier; the high temperature gasifier being operative to function at temperatures of greater than or equal to about 450° F.; converting the carbon monoxide into carbon dioxide; and feeding the carbon dioxide to an synthesizing reactor to produce an oil rich algae.

### CROSS-REFERENCE TO RELATED APPLICATIONS

This application is a continuation in part of application Ser. No. 11/680,704 filed on Mar. 1, 2007, which claims the benefit of application Ser. No. 11/627,403 filed on Jan. 26, 2007, which claims the benefit of application Ser. No. 11/621,801 filed on Jan. 10, 2007, which claims the benefit of application Ser. No. 11/620,018 filed on Jan. 4, 2007, the entire contents of which are incorporated herein by reference.

### BACKGROUND

This disclosure relates to a method and a system for converting waste into energy. More specifically this disclosure relates to a method and to a system for converting carbonaceous materials including waste into energy.

Human beings generate large amounts of waste across the world everyday. In addition to the waste generated by everyday human activity, waste is also generated by industrial and manufacturing activity. Some of this waste is discharged into landfills, while other portions of this waste are discharged into the ground or into bodies of water such as streams, rivers and oceans. This waste is often hazardous to living beings. For example, runoff from landfills can get into ground water and contaminate it. Toxic wastes from industrial facilities can also contaminate the atmosphere as well as bodies of water such as streams, rivers and oceans. Gases discharged from industrial facilities often contain carbon dioxide, which is believed to contribute to global warming.

In addition, waste that was stored in landfills is often burnt in order to reduce its volume so that the landfill can be used for extended periods of time. However, the burning of waste matter often produces carbon dioxide. This is now considered to be environmentally hazardous because the carbon dioxide contributes to global warming. Heavy metals, dioxins and furans, which are generally considered to be toxins are also produced.

It is therefore desirable to be able to dispose waste matter that is generated by living beings in a manner that protects the environment. It is further desirable to recycle this waste into energy so as to reduce the cost of energy generation as well as to recover the energy expended in the manufacturing of products that end up as waste. It is also desirable to generate energy from waste that is discarded to landfills in a manner that does not further degrade the environment, and that can produce fuel for combustion engines.

### SUMMARY

Disclosed herein is a system comprising a high temperature gasifier; the high temperature gasifier being operative to function at temperatures of greater than or equal to about 450° F.; and a synthesizing reactor in fluid communication with the high temperature gasifier; the synthesizing reactor being operative to convert carbon dioxide into another form of carbon.

Disclosed herein too a method comprising gasifying a waste stream to produce a composition that comprises syngas; the syngas comprising carbon monoxide and hydrogen; the gasifying being conducted in a high temperature gasifier; the high temperature gasifier being operative to function at temperatures of greater than or equal to about 450° F.; converting the carbon monoxide into carbon dioxide; and feeding the carbon dioxide to an synthesizing reactor to produce an oil rich algae.

### BRIEF DESCRIPTION OF FIGURES

FIG. 1 is an exemplary depiction of a system that comprises a high temperature gasifier in communication with a synthesizing reactor;

FIG. 2 is an exemplary depiction of the carbon loop that comprises a plasma gasifier (that serves as the high temperature gasifier), an oxygenation reactor and the synthesizing reactor;

FIG. 3 is one exemplary depiction of the hydrogen loop that comprises the carbon oxygenation reactor and a device that can generate energy;

FIG. 4 is an exemplary embodiment of the system that comprises an algae bioreactor and a hydrogen separator in fluid communication with a plasma gasifier;

FIG. 5 is another exemplary embodiment of the system that comprises a water gas shift reactor in fluid communication with a plasma gasifier;

FIG. 6 is an exemplary depiction of the system that comprises a closed carbon loop and a closed hydrogen loop that contains a hydrogen engine that is in fluid communication with the water gas shift reactor;

FIG. 7 is an exemplary depiction of the system wherein the hydrogen loop comprises a heat recovery electricity generator;

FIG. 8 depicts an exemplary carbon loop that is similar to that shown in the FIG. 7, but with a methane gas supply that is mixed with the hydrogen;

FIG. 9 is an exemplary depiction of the system that comprises a storage tank and water separator located downstream of a syngas engine;

FIG. 10 is an exemplary depiction of the system that comprises a syngas boiler or syngas engine and water separator as well as a heat recovery electric generator;

FIG. 11 is a depiction of the system that comprises an outer loop and an inner loop; the outer loop comprises a heat recovery electricity generator or a syngas engine;

FIG. 12 is a depiction of the system that comprises an outer loop and an inner loop; the outer loop; the inner loop transfers carbon dioxide and released oxygen from the algae bioreactor to the syngas engine or syngas boiler;

FIG. 13 depicts one exemplary method of nighttime operation that comprises the storage and sequestration of carbon dioxide;

FIG. 14 depicts another embodiment of an open loop for night-time operation; in the embodiment depicted in the FIG. 13, the syngas produced by the plasma gasifier can be used as a feedstock in a Fischer-Tropsch process;

FIG. 15 depicts another embodiment directed to night-time operation where hydrogen is maintained during daytime operation as a reserve fuel; and

FIGS. 16-18 are exemplary depictions demonstrating the performance of the system with various components.

### DETAILED DESCRIPTION

It is to be noted that as used herein, the terms “first,” “second,” and the like do not denote any order or importance, but rather are used to distinguish one element from another, and the terms “the”, “a” and “an” do not denote a limitation of quantity, but rather denote the presence of at least one of the referenced item. Furthermore, all ranges disclosed herein are inclusive of the endpoints and independently combinable.

Furthermore, in describing the arrangement of components in embodiments of the present disclosure, the terms “upstream” and “downstream” are used. These terms have their ordinary meaning. For example, an “upstream” device as used herein refers to a device producing a fluid output stream that is fed to a “downstream” device. Moreover, the “downstream” device is the device receiving the output from the “upstream” device. However, it will be apparent to those skilled in the art that a device may be both “upstream” and “downstream” of the same device in certain configurations, e.g., a system comprising a recycle loop.

The terms syngas engine electricity generator, syngas boiler electricity generator, hydrogen engine electricity generator, hydrogen boiler electricity generator are intended to indicate that an engine that utilizes syngas, hydrogen, a combination of hydrogen and a second fuel or a combination of syngas with a second fuel are used to drive an electricity generator. The engine is in communication with the electricity generator. The second fuel can comprise hydrocarbons such as methane, gasoline, diesel, or the like.

The waste stream fed to the high temperature gasifier is sometimes referred to as the feedstock or the feed stream.

Disclosed herein is a method and a system for converting carbonaceous materials, including hydrocarbons and carbohydrates and specifically waste into useful energy. The waste can be derived from municipal solid waste (MSW) e.g., landfill waste or can be obtained directly from waste streams of industrial or manufacturing facilities. The system advantageously comprises a high temperature gasifier for breaking down carbonaceous materials, which specifically include waste, into its basic elements and compounds. The basic elements and compounds comprise hydrogen and carbon monoxide. These products are then fed to reactors that are advantageously arranged to generate useful energy while at the same time minimizing carbon dioxide emissions out of the system (e.g., into the atmosphere). In one exemplary embodiment, the synthesizing reactors are arranged in a closed loop so as to reduce the emission of byproducts out of the system to nearly (substantially) zero.

The system has the ability to be self-sustaining. In other words, while producing oxygen gas, it can also produce hydrogen and vegetable oil in the form of oil rich algae. The energy can be derived from waste matter that is acquired from municipal waste or from the waste streams of industrial or manufacturing facilities. The system can generate energy in an amount of approximately 2925 kilowatt hour per ton of municipal waste when running at 40% efficiency with a syngas engine and/or 30% efficiency running in a heat recovery mode.

In one embodiment, the system comprises a carbon loop and a hydrogen loop. The carbon loop comprises the high temperature gasifier, an oxygenation reactor and a synthesizing reactor. The high temperature gasifier facilitates the conversion of waste into syngas, which comprises hydrogen and carbon monoxide. The carbon monoxide is converted into carbon dioxide in the oxygenation reactor. The carbon dioxide is converted into another form of carbon in the synthesizing reactor. In one embodiment, the carbon dioxide is converted to oil rich algae in the synthesizing reactor.

The oil rich algae can then be fed back to the plasma gasifier to generate additional hydrogen and carbon monoxide. The system can be operated in a manner that permits the carbon loop to contain a substantially constant amount of carbon. In other words the system is operated in such a manner that the average amount of carbon present in the waste, the carbon monoxide, the carbon dioxide and the oil rich algae is kept substantially constant through out the process. In order to continually harvest the oil rich algae, waste or other carbonaceous material of equal carbon weight can be substituted in the high temperature gasifier feedstock input.

The hydrogen loop uses hydrogen to generate energy. The hydrogen loop comprises the oxygenation reactor and an energy-generating device. The hydrogen is used to generate energy by feeding it to energy-generating devices such as a hydrogen engine electricity generator, a hydrogen boiler, syngas engine, syngas boiler, or the like. In one embodiment, these energy-generating devices generally use hydrogen or syngas generated in the high temperature gasifier and oxygen generated in the synthesizing reactor to generate energy. Steam is generated as a byproduct in the energy generating device that can be fed back to the oxygenation reactor.

In one embodiment, the system can be arranged to have an inner loop and an outer loop. The inner loop comprises the synthesizing reactor and is generally used to provide carbon dioxide and oxygen. Carbon dioxide that cannot be utilized in the inner loop is transferred back to the outer loop. Oxygen generated in the synthesizing reactor in the inner loop (can be separated from the carbon dioxide) and is used with the hydrogen generated in the high temperature gasifier to power an energy generation device such as for example a hydrogen engine, a syngas engine, a hydrogen engine electricity generator, a hydrogen boiler, or a fuel cell.

The outer loop is generally used to transport carbon in various forms. This transportation of carbon serves as a means of facilitating the gathering, transporting and harvesting of hydrogen and releasing of oxygen generated by the algae bioreactor during photosynthesis.

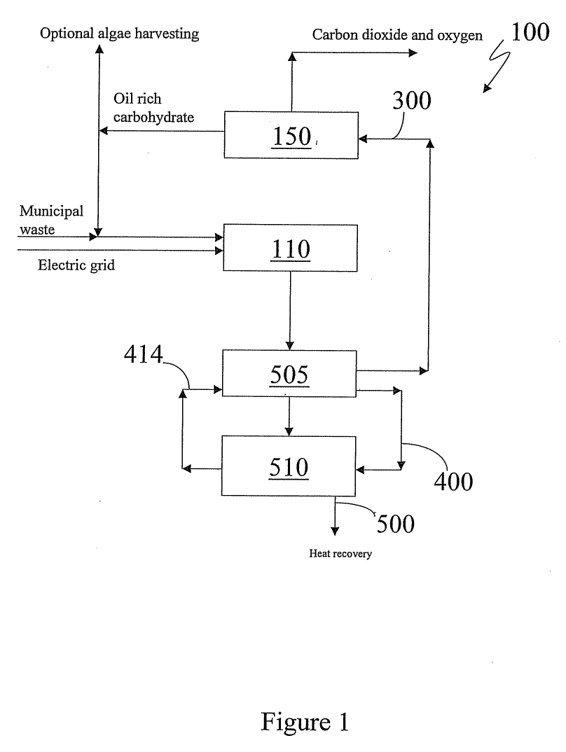

With reference now to the FIG. 1, the system 100 comprises a high temperature gasifier 110 that is in communication with an oxygenation reactor 505 (hereinafter carbon oxygenation reactor since it generally oxidizes carbon), and a synthesizing reactor 150. In one embodiment, the carbon oxygenation reactor 505 can be a water gas shift reactor. Other examples of carbon oxygenation reactors are catalytic converters, syngas boilers, syngas engines, and the like. The carbon oxygenation reactor 505 is generally located downstream of the high temperature gasifier 110 and is in fluid communication with it.

As can be seen in the FIG. 1, the carbon oxygenation reactor 505 is in fluid communication with a device 510. The device 510 can comprise a single or multiple pieces of equipment and can be a device that is operative to extract hydrogen, or that can use hydrogen for generating energy, or is a combination of both. Examples of the device 510 are a water gas shift reactor, a hydrogen separator, a hydrogen engine electricity generator, a hydrogen engine, a hydrogen boiler, or the like, or a combination comprising at least one of the foregoing devices.

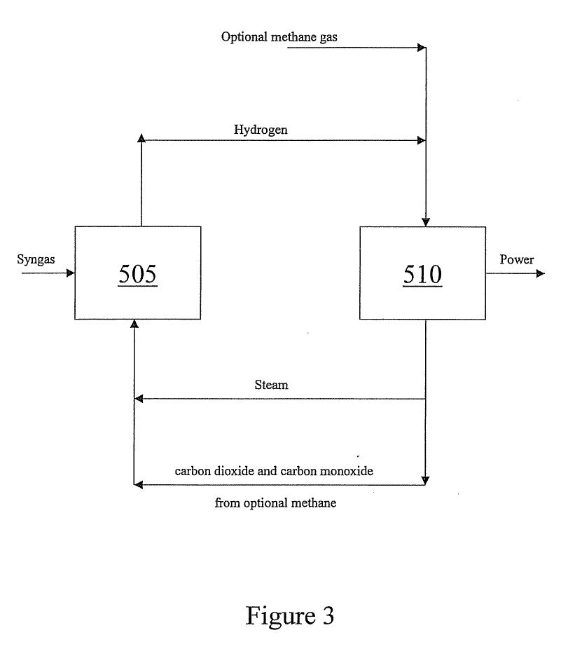

In one embodiment, the device 510 can be a part of a hydrogen loop 400 (depicted and described in the FIG. 3 below) that comprises a water gas shift reactor in addition to the device 510. In one embodiment, the device 510 is a hydrogen oxygenation reactor. Examples of hydrogen oxygenation reactors include a hydrogen boiler, a hydrogen engine, a fuel cell, or the like, all of which combine hydrogen with oxygen. The heat released by this exothermic process can be used to generate useful energy. Heat can be recovered from the device 510 using a heat recovery system 500.

In the FIG. 1, an exemplary hydrogen loop is shown to depict the carbon oxygenation reactor 505 in fluid communication with the device 510. It is to be noted that the hydrogen loop is optional. If no energy is to be generated from the hydrogen, then the hydrogen loop may be discontinued or avoided. As will be seen, the choice of a hydrogen oxygenation reactor as a part of the system depends upon the desired functioning of the system.

The synthesizing reactor 150 is located down stream of the high temperature gasifier 110 and carbon oxygenation reactor 505 and is used to absorb carbon dioxide and release oxygen. Carbon dioxide unabsorbed in the synthesizing reactor 150 may be released as a byproduct. It is generally desirable that the amount of oxygen released by the synthesizing reactor be of similar proportions to the carbon dioxide absorbed. Algae is one of the fastest growing plants on earth and for every ton of municipal solid waste processed, can absorb approximately 1900 pounds (lbs) of carbon dioxide and releases approximately 1600 lbs of oxygen.

In some embodiments, the synthesizing reactor is an algae bioreactor, a photo plankton reactor, an enzyme reactor, a bacterial reactor, or the like, or a combination comprising at least one of the foregoing synthesizing reactors. Algae bioreactors are generally superior to other synthesizing reactors, and function by photosynthesizing carbon dioxide with water in the presence of sunlight. This however does not preclude the use of other synthesizers that do not use photosynthesis in lieu of or in conjunction with algae bioreactors where desirable. In another embodiment, the synthesizing reactor may function during the night-time by using artificial lighting (e.g., grow-lights).

In one embodiment, the synthesizing reactor is part of a carbon loop 300. While the carbon loop 300 in the FIG. 1 has been depicted as a closed loop it may also be an open loop if desired. The system also may comprise a number of optional storage tanks (not shown) that can be used to store gases produced in either the high temperature gasifier 110 or in the device 510 till needed. These storage tanks can be used to smooth out demands in the production cycle. Other devices such as for example, valves, pumps, scrubbers, nozzles, and the like, can be employed in the system 100 where useful.

It is generally desirable for the high temperature gasifier 110 to operate in an oxygen depleted atmosphere at a temperature that is effective to break down the carbonaceous feed stream such as municipal waste into its basic elements and compounds, such as, for example carbon monoxide and hydrogen. Examples of high temperature gasifiers 110 are plasma gasifiers, oxygen injection gasifiers, Bessemer converters, molten metal gasifiers, or the like, or a combination comprising at least one of the foregoing high temperature gasifiers. In order to achieve this, it is generally desirable for the high temperature gasifier 110 to operate at temperatures of about 450° F. to about 50,000° F. Within this range, it is desirable for the high temperature gasifier 110 to operate at a temperature of greater than or equal to about 800° F., specifically a temperature of greater than or equal to about 1,200° F., specifically a temperature of greater than or equal to about 1,500° F., specifically a temperature of greater than or equal to about 1,800° F., specifically a temperature of greater than or equal to about 2,000° F., specifically a temperature of greater than or equal to about 2,200° F., specifically a temperature of greater than or equal to about 3,000° F., specifically a temperature of greater than or equal to about 4,000° F., specifically a temperature of greater than or equal to about 10,000° F., specifically a temperature of greater than or equal to about 20,000° F. and more specifically a temperature of greater than or equal to about 30,000° F. An exemplary high temperature gasifier 110 is a plasma gasifier that can operate at temperatures of greater than or equal to about 40,000° F.

Plasma gasifiers including molten metal gasifiers are generally superior to other gasifiers in that they heat up the feedstock electrically and independently of the oxygen input, while other gasifiers (e.g., fluidized bed, entrained flow, molten metal, rotating kiln, fixed bed), which use oxygen to heat up the feedstock are limited by the amount of oxygen that is used to convert carbon into carbon monoxide. This limits the operating temperature of the gasifier and renders the gasifier less able to produce hydrogen. Plasma gasifiers are able to release hydrogen from water by water shift reaction when producing carbon monoxide in the gasifier i.e., C+H₂═CO₂+H₂. As a result, plasma gasifiers are a good choice to break down municipal and industrial waste into their basic elements and compounds. They are also suitable because of the higher temperatures used to break down the wide array of unknown materials found in them. Exemplary plasma gasifiers can be obtained from Startech Corporation, Westinghouse Plasma and Integrated Environmental Technologies.

Other gasifiers are generally not able to break down all materials found in municipal waste because of their lower operating temperatures when compared with plasma gasifiers and therefore permit toxins to be released into the surroundings. This however does not preclude the use of the other types of gasifiers in conjunction with the plasma gasifiers, where desirable.

During processing in the high temperature gasifier, the hydrocarbons and the carbohydrates in the waste stream are converted into carbon monoxide and hydrogen. Small amounts of carbon dioxide can also be produced during the conversion of hydrocarbons and carbohydrates in the waste stream to carbon monoxide and hydrogen. These small amounts of carbon dioxide can be fed directly to the synthesizing reactor to facilitate photosynthesis by the algae.

The combination of carbon monoxide and hydrogen in the high temperature gasifier is sometimes referred to as “syngas”. As will be described later in this text, the combination of carbon monoxide and hydrogen from the high temperature gasifier is fed to the device 510 for further processing. Other products produced from the waste stream are molten solids such as, for example, base metals, silica, carbon and the like, can be drained off from the high temperature gasifier 110 in the form of a molten discharge and can be solidified upon cooling (not shown). These products can eventually be used for metal recovery, while other forms of low value slag obtained from the high temperature gasifier 110 can be used as building materials for industrial products. In one embodiment, the heat energy in these products can be recovered and used together with heat from other parts of the system to heat inlet water to a steam boiler or to evaporate a refrigerant gas to power a low temperature gas turbine engine or the like.

For the plasma gasifier to supply syngas (carbon monoxide and hydrogen), the supply of oxygen to the plasma gasifier has to be carefully controlled. Oxygen in the form of air, steam or water in the plasma gasifier initially increases the formation of carbon monoxide, and would then continue to be transformed into carbon dioxide. In the case where excess moisture in the feedstock makes it desirable to reduce the oxygen level in the plasma gasifier, this can be done by adding dry hydrocarbon (e.g., dry used tires, which adds carbon and hydrogen but not oxygen) to the feedstock. Tornado dryers and/or other moisture evaporation equipment (not shown) may also be employed to control the entry of moisture to the plasma gasifier.

The flow of feedstock through the gasifier can be increased without increasing the flow of carbon monoxide or carbon dioxide gases in the carbon loop. By maintaining the oxygen (and materials containing oxygen) supply (to the high temperature gasifier) constant and just sufficient to produce carbon monoxide but not carbon dioxide, the flow output will remain the same. Particles of carbon thus created in the syngas output by the extra carbon can be removed by filtration and/or electrostatic precipitation. Increased hydrogen will flow through the system with the increased feedstock throughput. To maintain a constant flow in the carbon flow loop, the weight of carbon particles removed can be balanced with additional feedstock, which contains carbon of same weight as that removed. Thus the amount of carbon in the carbon loop is substantially constant.

In one embodiment, it is desirable increase the flow of feedstock through the gasifier while reducing the amount of oxygen to the plasma gasifier. The waste products are converted to carbon upon being heated in the plasma gasifier. The carbon along with the hydrogen can be discharged from the plasma gasifier. The carbon and carbon particulates can be filtered out of the discharge from the plasma gasifier, while the hydrogen can be harvested. The harvested hydrogen can be used to generate energy.

In one embodiment, the amount of carbon in the carbon loop can be varied by an amount of about 5%, above or below the constant amount that can be theoretically established based on the weight and composition of the feedstock. In another embodiment, the amount of carbon in the carbon loop can be varied by an amount of about 10%, above or below the constant amount that can be theoretically established based on the weight of the feedstock.

Alternatively, by minimizing the oxygen input to the high temperature gasifier and filtering out the carbon particles from the high temperature gasifier syngas output, a minimum level of carbon gases (CO+CO₂) will circulate in the carbon loop. This will have the advantage of reducing the size of the synthesizing reactor (algae bioreactor) while maintaining low greenhouse gas emissions. Hydrogen production will increase with the increase in feedstock flow to the to the high temperature gasifier.

With reference once again to the FIG. 1, the device 510 is located downstream of the high temperature gasifier 110 and the carbon oxygenation reactor 505 and receives the syngas from the high temperature gasifier 110. The device 510 can comprise a single unit or can comprise multiple units.

The carbon oxygenation reactor 505 produces hydrogen and carbon dioxide, of which hydrogen is provided to the device 510. As noted above, the device 510 can be a hydrogen oxygenation reactor which can encompass any of the following a hydrogen boiler, a hydrogen engine, a hydrogen separator, a fuel cell, a syngas engine, a syngas boiler, or the like, or a combination comprising at least one of the foregoing devices.

The carbon-oxygenation reactor (e.g., a water gas shift reactor) operates at elevated temperatures of about 1800° F. to about 2200° F. and combines steam with carbon monoxide derived from the high temperature gasifier 110 to produce carbon dioxide and hydrogen. This is described in reaction (I) below:

CO+H₂O(steam)→CO₂+H₂ (I)

When the high temperature gasifier 110 is a plasma gasifier, the high temperature of the gases obtained from the plasma gasifier makes the combination of the water gas shift reactor with the plasma gasifier very advantageous. At higher temperatures, the reaction depicted in the equation (I) is driven towards the right hand side of the equation. This results in greater conversions of carbon monoxide to carbon dioxide with greater production of hydrogen. If it is desirable to carry out the reaction shown in the equation (I) at a lower temperature, then catalysts that comprise transition metals or transition metal oxides can be used to catalyze the reaction.

The hydrogen generated in the water gas shift reactor can be harvested for use in a fuel cell, a hydrogen boiler or in a hydrogen engine, while the carbon dioxide can be directed to the synthesizing reactor 150. As noted previously, it is desirable for the synthesizing reactor to be an algae bioreactor. In a fuel cell, the hydrogen is reacted with oxygen to produce water and electricity, the latter of which is can be used to power an electric motor, if desired.

The hydrogen engine is an internal combustion engine that ignites hydrogen with oxygen in its combustion chambers. This can be used to drive an electric generator, a motor, or other devices that convert energy from one form to another. When a boiler is used, steam from the boiler can be used to drive a steam engine or turbine, which drives an electric generator or other energy generation device. In this embodiment, the exhaust gas from the combustion in the hydrogen engine comprises steam and can be recycled to the water shift gas reactor to facilitate the reaction (I) as detailed above. In another embodiments, the exhaust gas (from the hydrogen engine or boiler) that comprises steam can be condensed to yield clean water.

A hydrogen separator is used to separate hydrogen from carbon dioxide. In particular, it is desirable to separate hydrogen from carbon dioxide that is generated in the water gas shift reactor. In one embodiment, the hydrogen separator utilizes separation by gravity (hydrogen being lighter than the molecular weight of carbon dioxide) to separate hydrogen from carbon dioxide. In another embodiment, a membrane can be used to separate hydrogen from other elements and compounds that are present in hydrogen containing mixtures obtained from the system 100. The membrane used in the hydrogen separator may be an inorganic membrane. A suitable inorganic membrane can comprise ceramics. Combinations of membrane and gravity separation can be used to separate the hydrogen from carbon dioxide.

A syngas engine ignites the hydrogen and carbon monoxide gases with oxygen in the engine combustion chamber and can be used to drive and electric generator and other devices. The exhaust gases from this process are steam, carbon dioxide (and other inert gases). The carbon dioxide can be fed downstream towards the algae bioreactor after recovering heat energy for useful work. This is shown in the reaction (II) below:

Syngas+Oxygen+Heat Release→Carbon dioxide+Steam (II)

With reference to the FIG. 1, the carbon loop 300 comprises the high temperature gasifier 110, the carbon-oxygenation reactor 505 and the synthesizing reactor 150. The carbon loop 300 can vent outside the loop, all untransformed gases not absorbed in the algae bioreactor.

As noted above, the synthesizing reactor 150 consumes the carbon dioxide produced in the water gas shift reactor. The synthesizing reactor 150 is an optically transparent device that uses the carbon dioxide (CO₂) and sunlight to grow oil rich algae. The exposure of algae to sunlight, water and CO₂ facilitates photosynthesis. To grow the algae, CO₂ is fed into a series of optically transparent “bioreactors”, which are filled with green algae suspended in nutrient-rich water (hereinafter “soup”). The algae use the CO₂, along with sunlight and water, to produce sugars by photosynthesis, which are then metabolized into fatty oils and protein. As the algae grow and multiply, portions of the soup are withdrawn from each reactor and can be dried into cakes of concentrated algae. These can be crushed or repeatedly washed with solvents to extract the oil. The algal oil can then be converted into biodiesel through a process called transesterification, in which it is processed using ethanol and a catalyst. Enzymes can then be used to convert starches from the remaining biomass into sugars, which are fermented by yeasts to produce ethanol.

Algae bioreactors 150 use high absorption algae, which in the presence of sunlight or grow lights feed on carbon dioxide to become a valuable source of oil rich carbohydrate. The carbon dioxide that would have been exhausted to the atmosphere is now converted from a global warming pollutant to a useful feedstock that is rich in hydrogen as shown in the theoretical reaction (III) below:

3.95CO₂+3.95H₂O→C_{3.95}H_{7.90}O_{2.81}+4.52O₂ (III)

where oil is half carbohydrate and half hydrocarbon.

In general terms the transformation may be described as shown in the reaction (IV) below:

nCO₂+2nH₂O→(CH₂O)ₙ+nH₂O+nO₂ (IV)

where n is about 3 to about 1510, ATP is adenosine triphosphate and NADPH is nicotinamide adenosine dinucleotide phosphate. It is to be noted that the formula for many carbohydrates may also be written as Cₙ(H₂O)ₙ. Examples of suitable carbohydrates are glucose, ketoses, monosaccharides, disaccharides, oligosaccharides and polysaccharides.

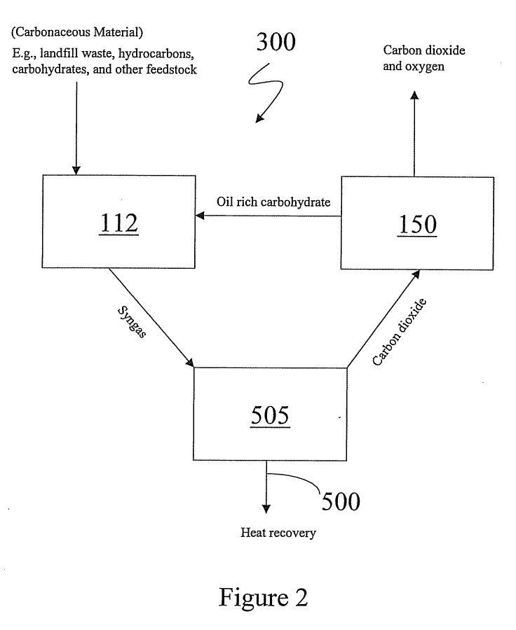

The carbon loop 300 can comprise the high temperature gasifier, the carbon oxygenation reactor and the synthesizing reactor. With regard to the FIG. 2, which comprises an exemplary embodiment, the carbon loop 300 comprises a plasma gasifier 112 (that serves as the high temperature gasifier), a carbon oxygenation reactor 505 and a synthesizing reactor 150. In one embodiment, the synthesizing reactor 150 is preferably an algae bioreactor, while the carbon oxygenation reactor is a water gas shift reactor. The plasma gasifier 112 operates at a temperature of greater than or equal to about 10,000° F. to form syngas. The syngas comprises substantially hydrogen and carbon monoxide. The syngas is fed to the carbon oxygenation reactor 505. In the carbon oxygenation reactor 505, steam reacts with the carbon monoxide to produce carbon dioxide and hydrogen. The hydrogen is separated from the carbon dioxide. The hydrogen is stored, fed to a hydrogen engine or a fuel cell to convert it into another useful form of energy or fed to a heat recovery boiler or other combustion or chemical device to extract heat. The carbon dioxide from the carbon oxygenation reactor 505 is fed to the synthesizing reactor 150 where it is consumed by algae in a photosynthesis reaction. To complete the carbon flow loop, the oil rich carbohydrate (algae) is fed back to the plasma gasifier 112. In this case, in order to maintain a constant flow in the carbon flow loop, the carbon dioxide vented to atmosphere or otherwise removed would need to be replaced with feedstock having the equivalent amount of carbon. An example of the carbon oxygenation reactor 505 is a water gas shift reactor depicted in some of the forthcoming figures.

The hydrogen loop 400 depicted in the FIG. 3 comprises the device 510 and the carbon oxygenation reactor 505. As explained above, the device 510 can be a hydrogen oxygenation reactor, examples of which are a hydrogen engine or a hydrogen boiler, a hydrogen engine electricity generator, a fuel cell, a syngas engine, a syngas boiler, and the like, all of which may be used to produce or convert hydrogen into useful energy and all of which are discussed below. It is to be noted that while the hydrogen engine or hydrogen boiler, hydrogen engine electric generators, fuel cell are not shown in the FIG. 3, they are depicted in other configurations below. These devices can be used individually or in combination with one another if desired. Some of these will be described in detail below. The carbon oxygenation reactor and the device 510 can be downstream of one another, i.e., they exist in a closed loop. However, as noted above, they can exist in an open loop as well.

As noted above the carbon oxygenation reactor can be a water gas shift reactor. The water gas shift reactor converts syngas into hydrogen and carbon dioxide. The water gas shift reactor can be fitted with a device for separating the hydrogen from the carbon dioxide if desired. The carbon dioxide is then fed to the synthesizing reactor 150 (not shown). The hydrogen is fed to the hydrogen oxygenation reactor, which ignites hydrogen with oxygen to generate energy. The hydrogen oxygenation reactor can be in communication with a generator or can act as a motor. Some of the steam, which is a product of combustion in the hydrogen oxygenation reactor, is fed back to the carbon oxygenation reactor 505 to facilitate the conversion of syngas.

In one embodiment depicted in the FIG. 3, a fuel may be fed to the hydrogen oxygenation reactor in addition to hydrogen. Examples of such fuels are natural gas, gasoline, diesel, or the like. In one embodiment, methane (CH₄) may be fed to the hydrogen oxygenation reactor in addition to the hydrogen. The mixture of methane and hydrogen ignites with oxygen in the hydrogen oxygenation reactor to generate steam plus carbon dioxide and possibly carbon monoxide, which are then fed back to the carbon oxygenation reactor 505. The carbon dioxide flows through the carbon oxygenation reactor 505 to become part of the carbon flow loop 300 (depicted in the FIG. 2 and in some of the figures described below). The carbon monoxide is converted to carbon dioxide in the carbon oxygenation reactor 505 to also become part of the carbon flow loop 300.

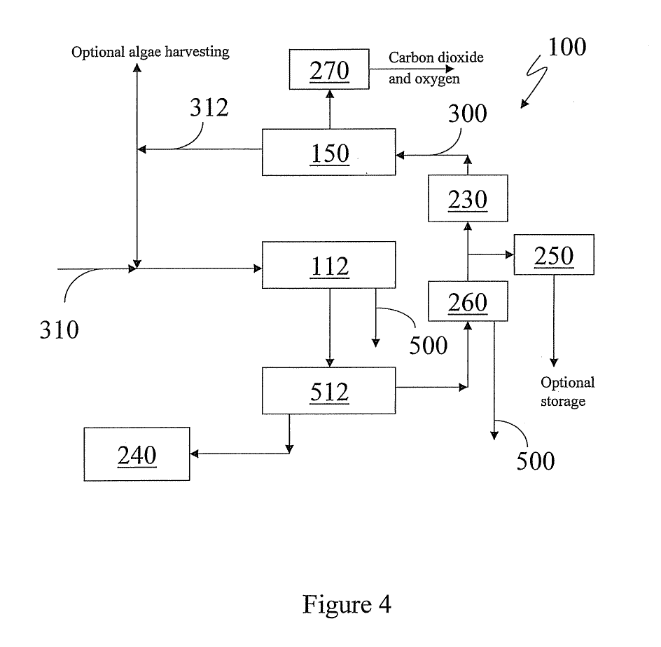

The system 100 is now depicted in various exemplary embodiments in the FIGS. 4 through 18. With reference now to the FIG. 4, an exemplary embodiment of the system 100 comprises a synthesizing reactor 150 and a hydrogen separator 512 in fluid communication with a plasma gasifier 112. In this exemplary embodiment, the plasma gasifier 112 serves as the high temperature gasifier described earlier. FIG. 4 is an exemplary depiction of the system 100 that is used to generate and store hydrogen and carbon dioxide without any energy generation. The system 100 therefore comprises a closed carbon loop 300 when the algae is fed to the gasifier 112 and an open loop when the algae is harvested. It does not have the hydrogen loop. The synthesizing reactor 150 and the hydrogen separator 512 are located down stream of the plasma gasifier 112 with the hydrogen separator 512 lying upstream of the synthesizing reactor 150. The system 100 also comprises a flow control valve 230, storage tanks 240 and 250, a catalytic converter 260 and a carbon dioxide sensor 270 disposed within the system as shown in the FIG. 4. Heat can be recovered by the heat recovery 500 at the plasma gasifier 112 and the catalytic converter 260.

In one embodiment, in one manner of operating the system 100 as depicted in the FIG. 4, a waste stream 310 (e.g., feedstock) along with optional oil rich carbohydrate 312 from the synthesizing reactor 150 is fed to the plasma gasifier 110 to generate the syngas. The hydrogen separator 512 receives the syngas from the plasma gasifier 112. The hydrogen separator 512 allows the small hydrogen molecules to pass through it. The rest of the syngas, comprising substantially carbon monoxide, then passes through to the catalytic converter 260 and is converted to carbon dioxide. The catalytic converter 260 generally comprises catalytic metals disposed on a suitable metal oxide to facilitate the conversion of carbon monoxide to carbon dioxide. Examples of catalytic metals are platinum and palladium. An exemplary metal oxide is aluminum oxide.

The carbon dioxide generated in the catalytic converter 260 is then received by the synthesizing reactor 150 where it is used to feed the algae. As noted above, an oil rich carbohydrate can be produced in the synthesizing reactor 150, a portion of which can be recycled to the plasma gasifier 112. A flow control valve (not shown) lying downstream of the catalytic converter 260 can be used to regulate the flow of carbon dioxide to the synthesizing reactor 150. Excess carbon dioxide can be stored in a storage tank 250 for use when desired, while hydrogen can be stored in a storage tank 240 and used when desired. The hydrogen stored in the storage tank 240 can be used to drive a hydrogen engine, a hydrogen boiler, a fuel cell or the like, or can be used in any other suitable application, if desired. A carbon dioxide sensor 270 detects the amount of carbon dioxide being vented to atmosphere. The flow control valve uses feedback from the carbon dioxide sensor 270 to control the CO₂ input to the synthesizing reactor 150, and control the CO₂ greenhouse gas emissions vented outside the system. This embodiment provides hydrogen but not electric power and therefore reduces the initial capital cost of the system.

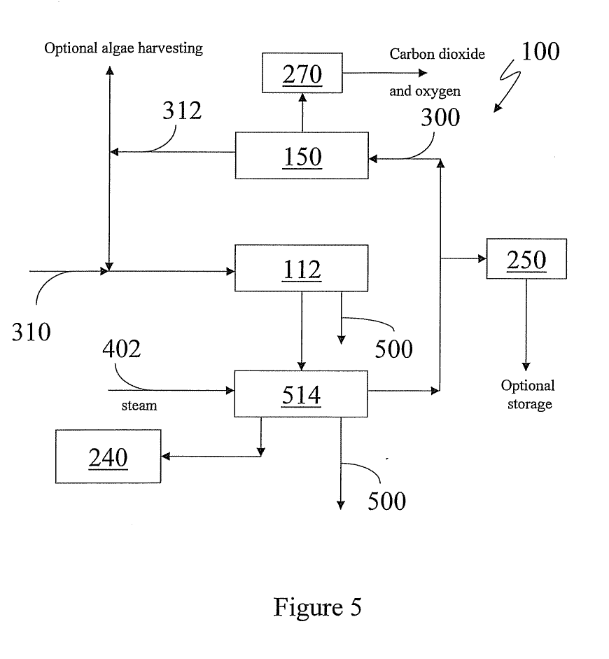

With reference now to exemplary embodiment depicted in the FIG. 5, the system 100 contains a water gas shift reactor 514 instead of the hydrogen separator 512 of the FIG. 4. The water gas shift reactor 514 can have a hydrogen separator (not shown) associated with it that can separate hydrogen from carbon monoxide. To increase the supply of hydrogen in the system, the carbon loop 300 depicted in the FIG. 5 is a closed loop and/or an optional open loop which comprises the plasma gasifier 112, the water gas shift reactor 514, and the synthesizing reactor 150. Storage tanks 240 and 250 perform the same functions as described above. The water gas shift reactor 514 uses steam obtained via line 402 and as described above converts the carbon monoxide to carbon dioxide and generates additional hydrogen. The carbon dioxide is fed via a hydrogen separator (not shown) to the synthesizing reactor 150, with additional carbon dioxide being stored in the storage tank 250. Hydrogen generated can be stored in the storage tank 240. The carbon dioxide detector 270 functions as described above.

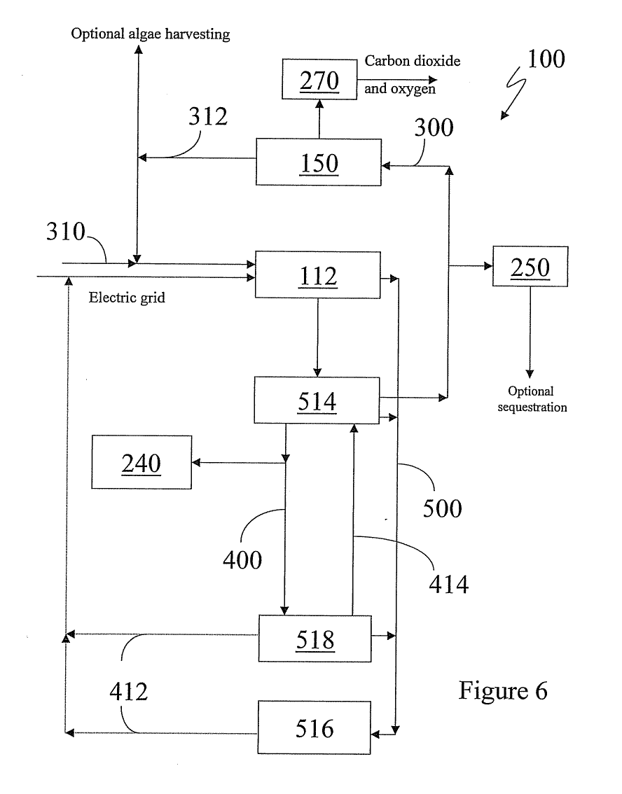

With reference now to the exemplary embodiment depicted in the FIG. 6, the system 100 comprises a hydrogen engine 518 that is in fluid communication with the water gas shift reactor 514. The system 100 as depicted in the FIG. 6 comprises both a closed carbon loop and/or an optional open loop 300 and a closed hydrogen loop 400. The carbon loop 300 depicted in the FIG. 6 comprises the plasma gasifier 112, the water gas shift reactor 514 and the synthesizing reactor 150. The hydrogen loop 400 is also a closed loop and comprises the water gas shift reactor 514 and a hydrogen engine 518. The hydrogen engine 518 provides steam and electric power. Recovered waste heat from the heat recovery system 500 is fed to heat recovery electric generator 516. Here refrigerant fluid is vaporized and used to fuel a low temperature turbine which drives an electric generator. Electricity from recovered heat is thus used to supply power via supply lines 412 to the electric grid.

The steam is fed to the water gas shift reactor (via pipe lines 414) to convert carbon monoxide to carbon dioxide, while the electricity generated by the hydrogen engine electricity generator 518 and the heat recovery electricity generator 516 can be used to power the plasma gasifier 112 via power supply lines 412. Other devices shown in the FIG. 6 such as the storage tanks 240 and 250, the synthesizing reactor 150 and the carbon dioxide detector 270 function as described above.

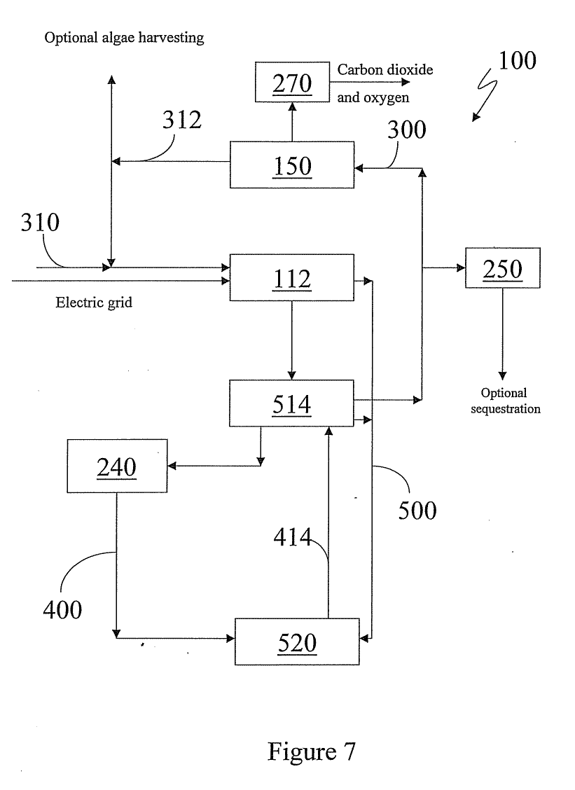

With reference now to the FIG. 7, the hydrogen loop comprises a storage tank 240 and a heat recovery boiler 520 disposed downstream of the water gas shift reactor 514. The storage tank 240 stores hydrogen, which is supplied to the heat recovery boiler 520. The heat recovery boiler 520 uses recovered heat to generate a boiled fluid (e.g., steam), supplemented by hydrogen combustion. A steam powered generator (not shown) in communication with the heat recovery boiler 520 supplies electric power. Steam is also fed to the water gas shift reactor 514 via pipelines 414 to convert carbon monoxide to carbon dioxide.

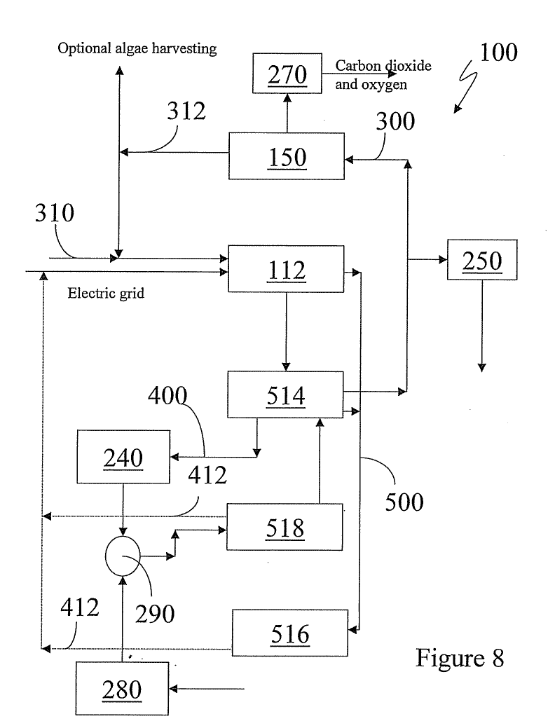

With respect to the FIG. 8, the carbon loop 300 is identical with that shown in the FIGS. 2, 5, 6 and 7. The hydrogen loop 400 comprises a hydrogen engine 518 that is in communication with a storage tank 240 that stores hydrogen. The hydrogen engine 518 can be used to generate electric energy as described above in reference to the FIG. 3. The storage tank 240 is upstream of the hydrogen engine 518. A second storage tank 280 stores methane. A mixing valve 290 lies downstream of the storage tank 240 and the storage tank 280. The storage tank 240 and the storage tank 280 are located on opposing sides of the mixing valve 290. A methane-hydrogen mixture can be fed from the respective storage tanks 280 and 240 to the hydrogen engine 518.

A heat recovery electricity generator 516 is located downstream of the plasma gasifier 112, the water gas shift reactor 514, and the hydrogen engine 518. The heat recovery electricity generator 516 provides steam and electricity in a manner similar to that described with reference to the FIG. 6. Thus electricity generated by the hydrogen engine 518 and the heat recovery electricity generator 516 can be used to power the plasma gasifier 112 as depicted by the lines 412. Steam from the hydrogen engine 518 can be supplied to the water gas shift reactor 514.

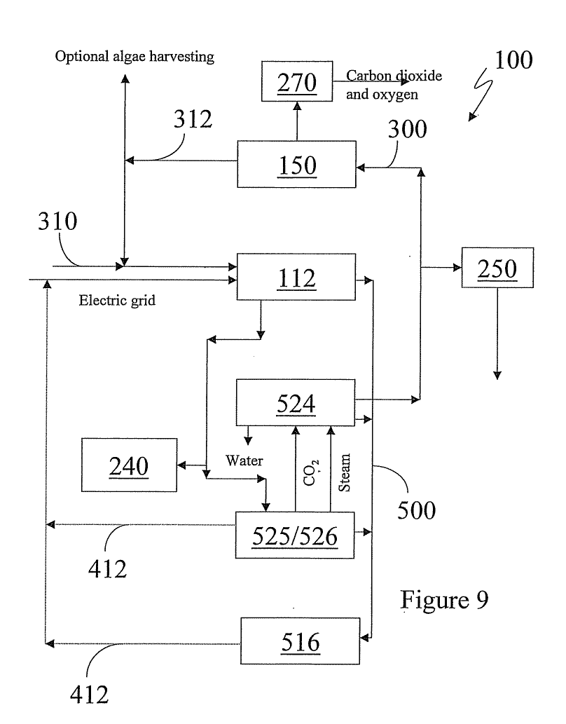

In the exemplary embodiment depicted in the FIG. 9, the carbon loop 300 comprises a plasma gasifier 112, a syngas boiler 525 or a syngas engine 526, a storage tank and water separator 524 and a synthesizing reactor 150, all of which are in fluid communication with one another. The syngas boiler 525 or syngas engine 526 is located downstream of the plasma gasifier 112. In one embodiment, the syngas engine 526 can be replaced by the syngas boiler 525. The storage tank and water separator 524 and the synthesizing reactor 150 are located downstream of the plasma gasifier 112. Syngas from the plasma gasifier 112 is discharged to the syngas engine 526. In the syngas engine 526 (as in the syngas boiler 525), both hydrogen and carbon monoxide are ignited with oxygen in the combustion chamber. Heat release is achieved by combining hydrogen with oxygen to produce steam and by combining carbon monoxide with oxygen to produce carbon dioxide. The syngas engine or the syngas boiler can be coupled with an electric generator to produce electricity.

The steam and carbon dioxide are discharged from the syngas engine 526 to the storage tank and water separator 524, where steam is condensed into water. The storage tank and water separator 524 also functions to separate water from the carbon dioxide. The carbon dioxide is discharged to the synthesizing reactor 150 where it is used to feed the algae. Unabsorbed carbon dioxide together with other unused gases is vented from the carbon loop. The device 240 is a storage tank that is used to store hydrogen. In one embodiment, the hydrogen stored can be that which is filtered from syngas.

Heat generated in the plasma gasifier 112, the syngas engine 526 and the storage tank and water separator 524 are fed to the heat recovery electricity generator 516, where electricity is generated. The heat recovery electric generator 516 operates at a low temperature by evaporating air conditioning type fluid so as to drive a low temperature turbine. The turbine can be used to drive an electric generator or other energy device for generating/releasing energy. As noted above, the syngas engine 526 and/or syngas boiler 525 can also be in communication with an electricity generating device that can generate electricity. The electricity generated by the heat recovery electricity generator 516 and the syngas engine 526 can be used by the plasma gasifier 112 as well as for other uses.

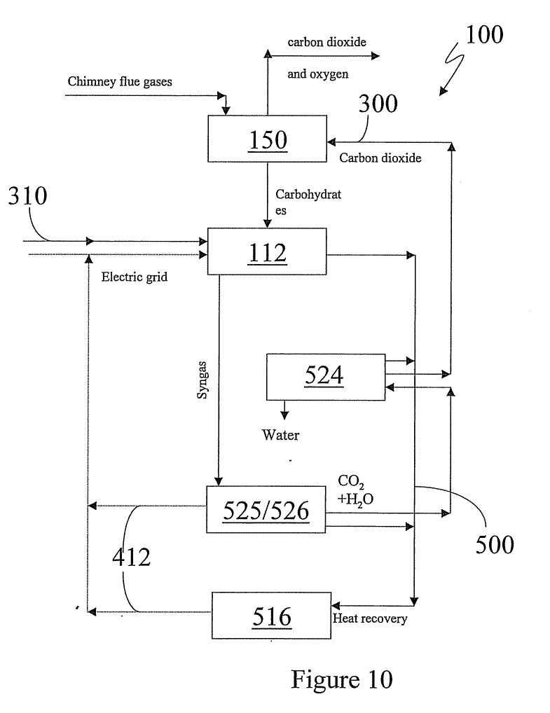

With reference now to the FIG. 10, the system 100 comprises a plasma gasifier 112 in fluid communication with a syngas engine 526. The syngas engine 526 is located downstream of the plasma gasifier 112 and receives syngas from the plasma gasifier 112. Carbon dioxide and steam generated in the syngas engine 526 are discharged to the storage tank and water separator 524. Carbon dioxide and water are separated in the storage tank and water separator 524. The carbon dioxide is discharged to the algae bioreactor 150 to produce oxygen and the oil rich algae. The oil rich algae is recycled to the plasma gasifier 112. Heat generated in the plasma gasifier 112 and the syngas engine 526 and the water separator 524 is fed to the heat recovery electricity generator 516. The heat recovery electricity generator 516 can provide steam to evaporate air conditioning fluid so as to drive a low temperature turbine. The turbine can be used to drive an electric generator or other energy device for generating energy.

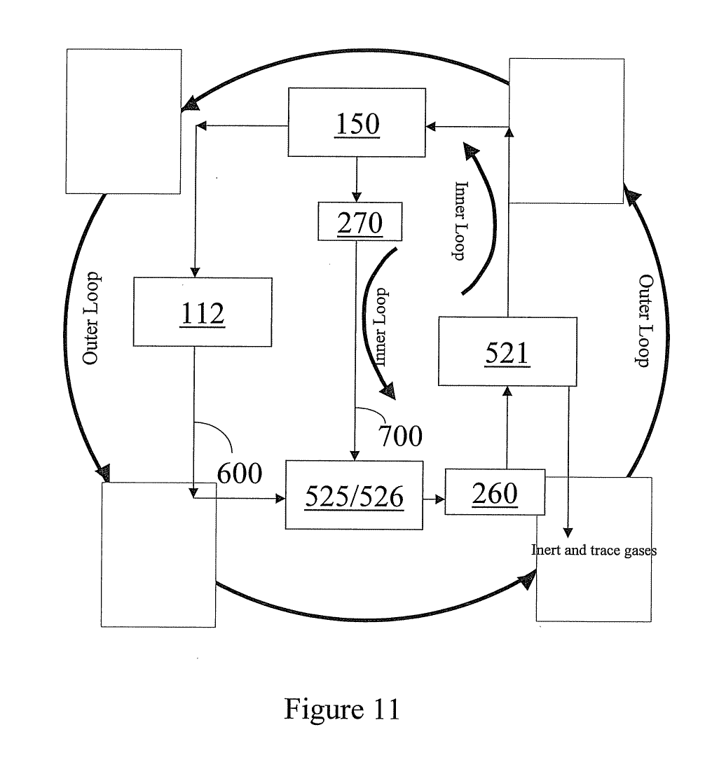

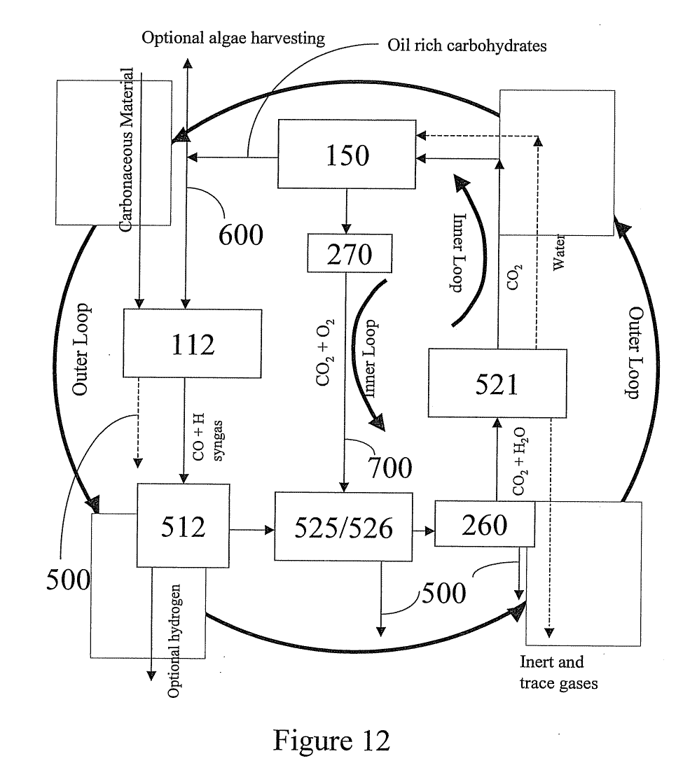

The system 100 can (as previously described) be in the form of a closed loop, which recirculates unabsorbed carbon dioxide instead of venting it. This takes the form of an outer loop 600 and an inner loop 700. As will be described, some components of the outer loop 600 are also used as components of the inner loop 700. With reference now to the FIGS. 11 and 12, the outer loop 600 comprising the plasma gasifier 112, an optional hydrogen separator 512, the syngas engine 526 and/or the syngas boiler 525, the catalytic converter 260, the storage tank and carbon dioxide separator 521 and the synthesizing reactor 150.

The carbon flow loop comprises carbon in various forms circulating through it. The various forms of carbon being carbonaceous feedstock, carbon black, carbon monoxide, carbon dioxide, carbohydrate (e.g., algae), and the like. Other carbonaceous materials such as methane may also be present in low volume. The carbon is balanced such that the carbon inflow to the loop equals the carbon outflow. In an open loop system, this means that the carbon flowing into the loop equals the carbon flowing out of the loop. In a closed loop system, the carbon flowing in equals the carbon flowing out. In these systems, the flow is limited by the flow capacity of the components in the system (e.g., the plasma gasifier and the algae bioreactor).

For example, when the closed loop system is operating normally, the recirculating carbon dioxide in the inner loop will be measured by the flow sensor 270 and be maintained at a set target flow rate. However, if conditions exist such that carbon dioxide absorption in the synthesizing reactor significantly drops, unabsorbed carbon dioxide flow in the system will increase. The flow sensor 270 reading will adjust the system such that the flow of carbon dioxide to the synthesizing reactor is reduced, as is the electric current flow to the plasma torch in the plasma gasifier. This in turn will reduce the carbon flow rate in the carbon loop, and keep the inflow of carbon (feedstock) equal to the outflow (algae). A similar situation would exist if the supply of feedstock to the plasma gasifier was reduced. In another embodiment, carbon could still flow in the carbon loop with no carbon inflow or outflow.

The system comprises carbon, oxygen and hydrogen flowing in loops, where the reactors in the respective loops transform the form of the elements and compounds into other substances. For example, the elements and compounds comprise hydrocarbons, carbohydrates, carbon monoxide, carbon dioxide, water, methane, carbon, hydrogen, oxygen and the like.

The loops can be open loops or closed loops. In one embodiment, in the open loops, materials entering the loops equals those leaving. In close loop operation, carbon, oxygen and hydrogen recirculate around the loops while other materials are vented out.

A system can have both open loops and closed loops. For steady state balanced operation, carbon, oxygen and hydrogen based materials flowing into the loops equals those leaving.

In one embodiment, in one method of operating as shown in FIG. 11, syngas produced by the plasma gasifier 112 is discharged to the syngas engine 526 or syngas boiler 525, which after combustion exhausts carbon dioxide and steam. The carbon dioxide and steam flows to the storage tank and carbon dioxide separator 521, where by increasing the pressure and/or reducing the temperature, the carbon dioxide gas becomes liquefied and settles below the lighter water at a much reduced volume. Inert and other gases are then vented, to prevent the accumulation of inert and other gases in the enclosed carbon flow loop. The carbon dioxide returns to a gas once the pressure is reduced. Pure carbon dioxide can now be fed to the synthesizing reactor 150 as desired. The carbon dioxide sensor located in the inner loop 700 measures the amount of carbon dioxide discharged to the inner loop by the synthesizing reactor 150. A flow control valve (not shown) situated downstream of the storage tank and carbon dioxide separator 521 uses this data to control the amount of carbon dioxide that is delivered to the synthesizing reactor 150.

The outer loop is generally used to transport carbon in its various forms. This transportation of carbon serves as a means of facilitating the gathering, transporting and harvesting of hydrogen generated by the algae bioreactor during photosynthesis. In the presence of sunlight, carbon dioxide and water, photosynthesis of the algae causes it to rapidly grow into an oil rich carbohydrate (carbon+hydrogen+oxygen). This carbohydrate can be fed back to the gasifier 112 in a closed loop or can be harvested in the open loop option. The harvested algae can be substituted with other carbon containing feedstock such as that available from MSW, landfill sewage or other waste, and fed to the plasma gasifier 112, where it is converted into syngas as described above.

During the combustion in the syngas engine or in the boiler, the syngas is converted into carbon dioxide and steam. It is then fed to the catalytic converter 260 to ensure conversion of any remaining carbon monoxide into carbon dioxide. The carbon dioxide is then transferred to the storage tank and carbon dioxide separator 521 or to other forms of containment, which stores and separates the carbon dioxide and water while venting the other gases. The carbon dioxide then flows to the flow control valve (not shown) and then onto the synthesizing reactor 150 as needed. The flow control valve supplies a regulated flow of carbon dioxide to the synthesizing reactor 150 by referencing the data supplied by the carbon dioxide sensor 270 in the inner loop to a target value.

The inner loop 700 comprises the carbon dioxide sensor 270, the syngas engine 526 and/or the syngas boiler 525, the catalytic converter 260, the storage tank and carbon dioxide separator 521 and the synthesizing reactor 150. In the inner loop, the carbon dioxide not digested by the algae in the synthesizing reactor 150, in addition to the oxygen released during photosynthesis are fed via the carbon dioxide sensor to the syngas engine 526. During combustion in the syngas engine 526, oxygen combines with the syngas to form carbon dioxide and steam, while the carbon dioxide passes through as an inert gas. The carbon dioxide now becomes part of the outer loop. This provides an overall means of gathering, transporting and harvesting hydrogen without emitting carbon dioxide, a greenhouse gas, to the atmosphere.

In one embodiment, the system 100 of the FIGS. 11 and 12 can be used as a closed loop feedback control system. The synthesizing reactor 150 can be sized to match the carbon dioxide from the carbon oxygenation reactors during specified minimum climatic and weather conditions, light intensity, temperature conditions, or the like.

In one embodiment, it is desirable for the synthesizing reactor 150 to contain a sufficient mass of algae for carbon dioxide digestion. It is also generally desirable for the amount of carbon dioxide that is supplied to the algae bioreactor to be only sufficient to meet the desired absorption capability of the algae. The flow control valve measures the carbon dioxide flow rate in the inner loop and references this to a targeted value. The flow control valve can be a proportional, derivative, differential or similar device and would be suitable for the closed loop 700 system where it senses the error from a target and continuously corrects the amount of carbon dioxide being unabsorbed by the synthesizing reactor 150.

In another embodiment, in order to regulate the amount of carbon dioxide in the storage tank, a variable storage level may be used. This would occur if there were a need to store carbon dioxide generated in the night time when photosynthesis in the bioreactor relies on artificial lights (e.g., grow lights) 151 to activate photosynthesis. To accommodate reduced algae production, the dawn level of carbon dioxide will be at the high point and the dusk level at the low point. With the targeted contents of the storage tank defined in this manner, the level of carbon dioxide in the tank can be also monitored and referenced to the targeted values through the night. In other words, if the storage tank level is too high then the plasma gasifier output will need to be reduced. This will be accomplished by reducing the targeted amount of carbon dioxide fed to the inner loop by the algae bioreactor. This also calls for reduced electric current flow to the plasma gasifier torch.

With reference once again to the FIGS. 11 and 12, it is desirable to maintain a chemical balance for the synthesizing reactor 150. The algae bioreactor operation can be described as follows in the equation (V) below:

Carbon fed to algae bioreactor−carbon to the inner loop=algae bioreactor output carbon (V)

In one embodiment, it is desirable for the amount of carbon dioxide generated in either the water gas shift reactor 514 or the syngas engine 526 to be completely consumed in the synthesizing reactor 150. In a similar manner, it would be desirable for the carbohydrate output from the algae bioreactor to be completely consumed in the plasma gasifier 112 to produce an amount of carbon monoxide that would be converted to carbon dioxide that can then be completely consumed in the algae bioreactor. However if the synthesizing reactor 150 is unable to perform photosynthesis at night, the plasma gasifier is generally designed to run all day while the algae bioreactor has to be sized to function during daylight hours only. In one embodiment, the algae bioreactor can use artificial lighting (grow-lights) in order to function at night.

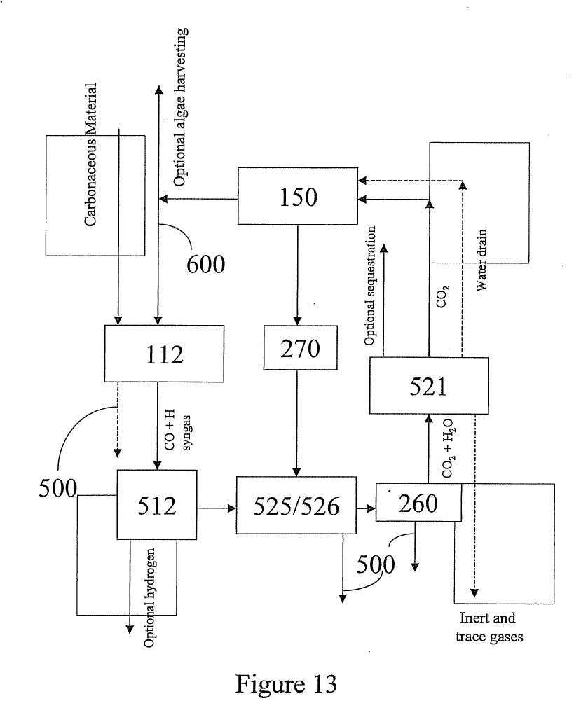

FIG. 13 depicts one method of nighttime operation that comprises storage and sequestration of carbon dioxide. During night time operation when reduced photosynthesis take place with artificial lights, a reduced amount of carbon dioxide is fed to the algae bioreactor. The remainder is liquidized and stored for later use.

In one embodiment, living organisms (e.g., algae, plankton, bacteria, enzymes) that do not use light can be used in the synthesizing reactor. As can be seen in FIG. 13, the plasma gasifier 112, the hydrogen separator 512, the syngas engine 526, the catalytic converter 260, carbon dioxide sensor 270, storage tank and carbon dioxide separator 521 and the synthesizing reactor 150 form a closed loop, with the syngas engine 526 (or syngas boiler 525) being located downstream of the plasma gasifier 112. The synthesizing reactor 150 is located downstream of the storage tank and carbon dioxide separator 521. A flow control valve (not shown) is located downstream of the storage tank and carbon dioxide separator 521. All of the aforementioned components in FIG. 13 are in fluid communication with one another either directly or indirectly.

With reference now to FIG. 13, a combination of landfill, waste and oil rich carbohydrate can be fed to the plasma gasifier 112 to produce carbon monoxide and hydrogen. A portion of the hydrogen from the plasma gasifier 112 is separated by the hydrogen separator 512, while the remainder of the syngas is discharged to the syngas engine 526 to produce carbon dioxide and steam. These are separated in the storage tank and carbon dioxide separator 521. The carbon dioxide passes through the storage tank and carbon dioxide separator 521 and on to the synthesizing reactor 150. Heat from the products of the plasma gasifier 112, the syngas engine 526, the syngas boiler 525 and the catalytic converter 260 may be used for heat recovery. Solids such as the silica may be removed from the plasma gasifier 112.

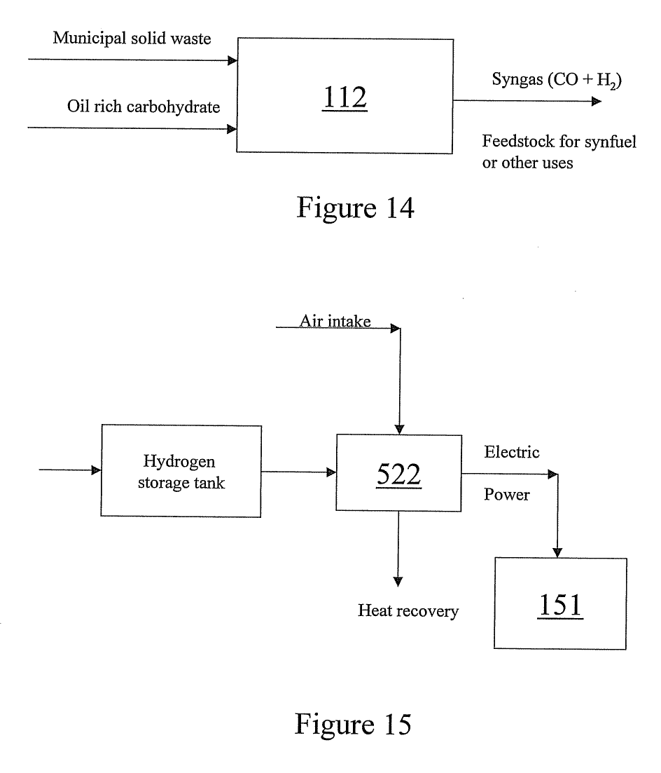

For night-time operation two open loop operating modes can be used and though they are listed individually in the FIGS. 14 and 15, they are mutually exclusive of each other and may be used either separately or in conjunction with one another when desired.

The FIG. 14 depicts another embodiment of an open loop for night-time operation. In the embodiment depicted in the FIG. 14, the syngas produced by the plasma gasifier 112 can be used as a feedstock in a Fischer-Tropsch process. The Fischer-Tropsch process is a catalyzed chemical reaction in which carbon monoxide and hydrogen are converted into liquid hydrocarbons of various forms. Exemplary catalysts used are based on iron and cobalt. This process is used to produce a synthetic petroleum substitute, generally from coal, natural gas or biomass, for use as synthetic lubrication oil or as synthetic fuel.

FIG. 15 depicts another embodiment directed to night-time operation. Hydrogen is maintained during daytime operation as a reserve fuel and the algae bioreactor can operate during the night using artificial lighting for photosynthesis. A hydrogen generator 522 or fuel cell 530 (not shown) can operate using the reserve hydrogen fuel supply to allow electrical power to be generated without emitting carbon dioxide to the atmosphere. Other energy storage devices can also be used in conjunction with the system depicted in the FIG. 15. For example, battery storage or other chemical, potential energy and kinetic energy devices can also be used to provide night-time electrical power.

In one embodiment, heat generated from the plasma gasifier, the gasifier molten discharge (e.g., base metals, silica, and the like), the catalytic converter, the syngas engine, and the like can be recovered and used for the cogeneration of energy. To improve low temperature heat recovery, the Kalina cycle, Ormat, or low temperature turbines can be used. These units use waste heat to evaporate a refrigerant. These can be used to power a low temperature gaseous turbine engine, which drives a generator to supplement the electric power provided by the generator engine. Specific use of these technologies will depend upon the size of the system and the emphasis placed on heat recovery.

This method and system is advantageous in that there are minimal emissions to the environment. The system uses landfill waste that under normal circumstances generally produces methane and/or carbon dioxide, both or which are greenhouse gases. The system can reduce the amount of carbon dioxide that is emitted into the atmosphere by an amount of up to about 50%, specifically by an amount of up to about 70%, specifically by an amount of up to about 90%, specifically by an amount of up to about 95%, specifically by an amount of up to about 99%, and more specifically by an amount of up to about 99.9%, when compared with a landfill, an incinerator or a gasifier that is not in communication with a device that uses the carbon.

The system is also advantageous in that it provides a means for gathering, transporting and harvesting hydrogen. In an exemplary embodiment, the hydrogen generated from the waste streams can be used to produce electricity. Thus matter that is normally discarded can be used to recover energy. The system can be utilized for power generation in power generation plants. Power generation plants can now be in communication with waste collection sites.

The system is also advantageous in that both hydrogen and oxygen generated in the system can be fed to an energy generating device such as for example, a fuel cell, a hydrogen engine, a hydrogen boiler electricity generator, or the like to produce energy. The system can thus be self-contained.

The system is also advantageous in that the amount of carbon circulating in the carbon loop can be maintained to be substantially constant. In one embodiment, the amount of carbon circulating in the carbon loop can vary by an amount of up to ±5 weight percent of a constant amount of carbon that circulates in the loop. In another embodiment, the amount of carbon circulating in the carbon look can vary by an amount of up to ±10 weight percent of a constant amount of carbon that circulates in the loop. In yet another embodiment, the amount of carbon circulating in the carbon look can vary by an amount of up to ±20 weight percent of a constant amount of carbon that circulates in the loop.

This disclosure is further described by the following non-limiting examples:

### EXAMPLES

### Example 1

This example is a paper example that demonstrates the potential savings and the potential energy that can be generated by using municipal landfill waste. The energy content of an exemplary sample of municipal waste is shown in Table 1 below.

TABLE 1

| | Approximate |
|---|---|
| | Energy Content |
| | (British thermal |
| Reference Materials | units/pound) |
| Municipal Solid Waste (MSW) | 4,000-7,000 |
| Wood | 8,000 |
| Coal | 9,000-12,000 |
| Algae | 9,000 |

One ton of municipal solid waste produces approximately 520 lbs of carbon, which as indicated above is a reference amount that remains relatively constant in the loop in the various forms. The 520 lbs of carbon in the municipal solid waste when transformed with oxygen produces carbon monoxide, carbon dioxide and carbohydrate (oil rich algae). The amount of carbon monoxide, carbon dioxide and carbohydrate from one ton of MSW are listed below in Table 2.

TABLE 2

| | Approximate Quantity |
|---|---|
| Component | (lbs) |
| Carbon Monoxide (From MSW) | 1215 |
| Carbon Dioxide in outer loop | 1910 |
| Carbon dioxide in inner loop | 190 |
| Carbohydrate | 1100 |

For the purposes of this estimate, when municipal waste is gasified in a plasma gasifier, it is assumed that the various gases that will be produced in the gasifier are as follows: 50 percent hydrogen by volume; 40 percent carbon monoxide by volume, with the remaining 10 percent being other gasses, such as carbon dioxide and trace methane.

### Example 2

### Fuel Cell Charging

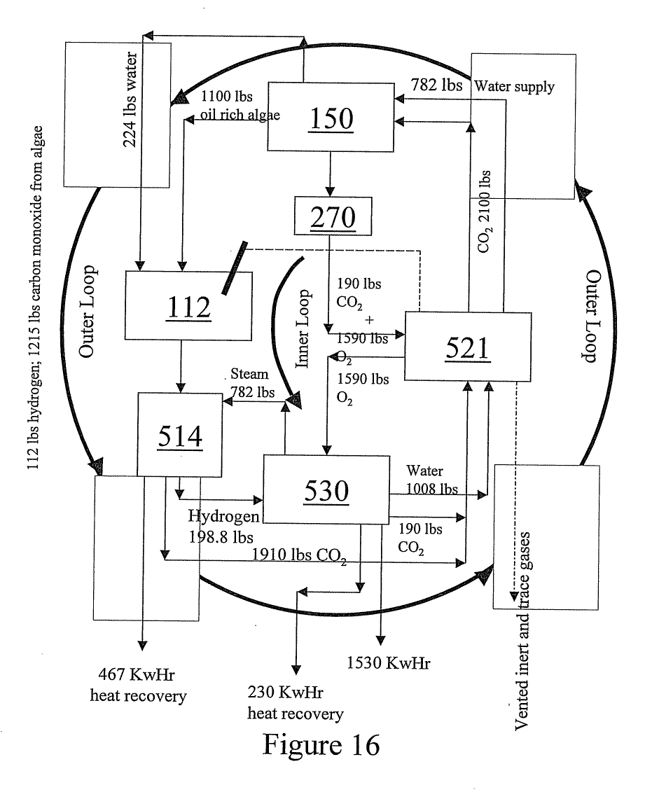

This is a paper example to demonstrate fuel cell charging. With reference to the FIG. 16, the inner loop 700 comprises the algae bioreactor 150, the carbon dioxide flow sensor 270, and fuel cell 530 and a storage tank and carbon dioxide separator 521 (which liquefies carbon dioxide and separates water and vents off other gases). The outer loop 600 comprises the high temperature gasifier 112, the hydrogen separator 514, water gas shift reactor 514, a fuel cell 530 and algae bioreactor 150 and the storage tank and carbon dioxide separator 521 (which liquefies carbon dioxide, separates water from the carbon dioxide and vents off other gases).

In this application, which is depicted in the FIG. 16, discharge from algae bioreactor 150 into the inner loop 700 contains only unabsorbed carbon dioxide and oxygen generated in the algae bioreactor 150 is discharged to a separate compartment in the storage tank and carbon dioxide separator 521. Here carbon dioxide is fed to the outer loop and oxygen to the fuel cell 530 for generating electricity.

Since no carbon exits the recirculating carbon flow loop, none needs to be added. Thus there is no feedstock input after the initial charge, just the recirculating carbon loop with oil rich algae being fed back to the plasma gasifier. With reference to the FIG. 16, 1,100 lbs of oil rich algae is fed to the plasma gasifier 112. This comprises 521 lbs of carbon. As noted above, in the Table 2, this is transformed into 1215 lbs of carbon monoxide in the plasma gasifier.112. This is then discharged into the water gas shift reactor 514 and transformed into 1910 lbs of carbon dioxide. The inner carbon loop feeds back 190 lbs of carbon dioxide. This equals 2100 lbs CO₂ fed to the algae bioreactor 150. The algae bioreactor absorbs 91% of this and produces 1100 lbs of oil rich algae.

The high temperature gasifier 112 produces 112 lbs of hydrogen from the algae and the water shift of algae oil. This is fed to the water gas shift reactor where the amount of hydrogen produced in conjunction with the conversion of carbon monoxide to carbon dioxide is increased to 198.8 lbs from 112 lbs. The fuel cell 530 transforms 198.8 lbs of hydrogen plus 1590 lbs of oxygen into 1789 lbs of water. 782 lbs of water are used by the water gas shift reactor 514. 782 lbs of water are used by the algae bioreactor 150 and 228 lbs are used by the plasma gasifier to convert algae to carbon monoxide and hydrogen.

From the fuel cell 530, the power output at 50% overall efficiency=1530 KwHr/Ton MSW. The heat recovery from the fuel cell 530 is 230 KwHr, while the heat recovery from the water gas shift reactor is 467 KwHr. The total power generated from the device depicted in the FIG. 16 is 2227 KwHr.

### Example 3

### Electric Power

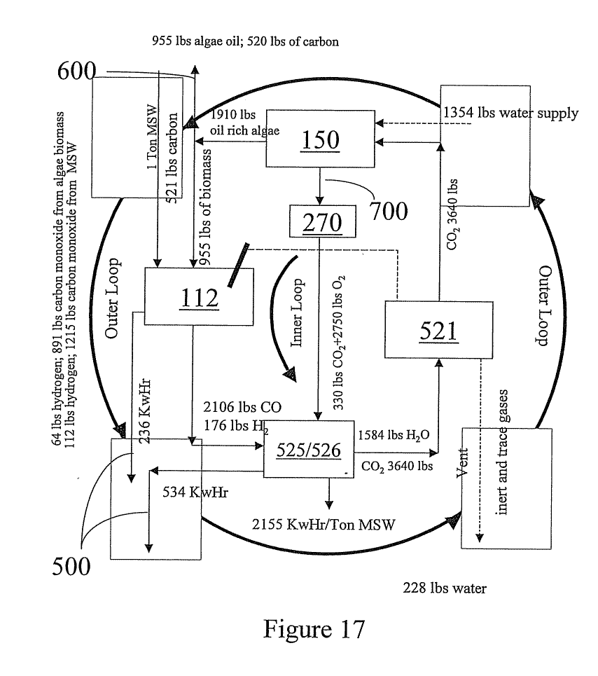

With reference to the FIG. 17, the inner loop 700 comprises the algae bioreactor 150, the carbon dioxide flow sensor 270, a syngas engine electric generator 525/syngas boiler electric generator 526 and a water storage and carbon dioxide separator 521 (which liquefies carbon dioxide and separates water and vents off other gases). The outer loop 600 comprises the high temperature gasifier 112, a syngas engine electric generator 525/syngas boiler electric generator 526 and a water storage and carbon dioxide separator 521 (which liquefies carbon dioxide, separates water from the carbon dioxide and vents off other gases).

With reference to FIG. 17, 2000 lbs of municipal solid waste (MSW) is fed to the plasma gasifier 112, together with 955 lbs of cellular biomass from the algae bioreactor 150. These contains 903 lbs of carbon. This is transformed into 2106 lbs of carbon monoxide in the high temperature gasifier 112 and 300 lbs of other gases, a portion of which is methane and carbon dioxide. The high temperature gasifier produces 176 lbs of hydrogen from the MSW and biomass. The syngas is fed to the syngas engine electric generator or syngas boiler electric generator. The syngas engine electric generator or syngas boiler electric generator transforms 2106 lbs of carbon monoxide from the syngas into 3310 lbs of carbon dioxide. The inner carbon loop feeds back 330 lbs of unabsorbed carbon dioxide to produce 3640 lbs CO₂, which is fed to the algae bioreactor 150. The algae bioreactor absorbs 91% of this and produces 1910 lbs of oil rich algae. Oil from the algae weighing 955 lbs is harvested from the system. The carbon content of the oil is 521 lbs. This equals the 521 lbs of carbon added to the system in the 2000 lbs of MSW. Carbon balance is thus achieved. The remaining 955 lbs of algae cellular biomass is fed back to the plasma gasifier. This provides 891 lbs of carbon monoxide and 64 lbs of hydrogen.

The syngas engine electric generator or syngas boiler electric generator transforms 176 lbs of hydrogen in the syngas into 1584 lbs of water. 1354 lbs of water is used by the algae bioreactor 150 and 230 lbs of water is used by the high temperature gasifier 112 this being equal to the hydrogen output in the form of water.

The following is a list of oxygen used for stoichiometric combustion:

1200 lbs of oxygen to transform carbon monoxide into carbon dioxide.
1408 lbs of oxygen to transform hydrogen into water.
2750 lbs is supplied by the algae bioreactor.
The following is a list of output energy:
Gasifier/syngas output=236 KwHr/Ton MSW @ 30% overall efficiency.
Power output from heat recovery=534 KwHr/Ton MSW @ 30% overall efficiency from engine exhaust
Power output from syngas combustion=2155 KwHr/Ton MSW @ 40% overall efficiency
Total power output=2925 KwHr/Ton MSW

### Example 4

### Methane Production with Closed Loop System

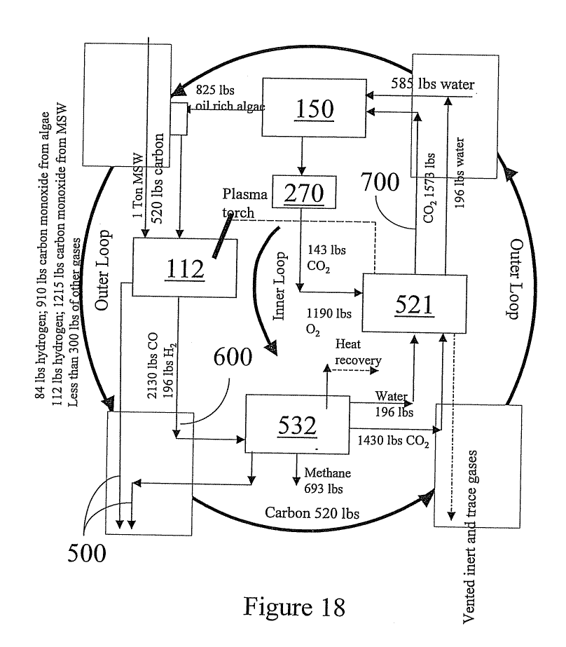

With reference to the FIG. 18, the inner loop 700 comprises the algae bioreactor 150, the carbon dioxide flow sensor 270, and the carbon dioxide separator 521 (which liquefies carbon dioxide, separates water from the carbon dioxide and vents off other gases). The outer loop 600 comprises the high temperature gasifier 112, the methanation plant 532, the carbon dioxide separator 521 and the algae bioreactor 150.

As noted above, 2000 lbs of municipal solid waste (MSW) together with 825 lbs of oil rich algae from the bioreactor 150 is fed to the plasma gasifier 112. This contains 521 lbs of carbon from the MSW and 390 lbs from the bioreactor. This is transformed into 2130 lbs of carbon monoxide in the plasma gasifier 112. This is then discharged into the methanation plant 532 and transformed into 693 lbs methane and 1430 lbs of carbon dioxide. The inner carbon loop then adds 143 lbs of carbon dioxide and 1190 lbs oxygen. This equals 1573 lbs CO₂ fed to the algae bioreactor 150. The algae bioreactor absorbs 91% of this and produces 825 lbs of oil rich algae.

The high temperature gasifier 112 produces 112 lbs of hydrogen from the municipal solid waste (MSW) and 84 lbs from the algae bioreactor input. This is fed to the methanation plant. 196 lbs of water are produced by the methanation plant

The output from the device of the FIG. 18 is 693 lbs of methane per ton of municipal solid waste.

### Sample Calculations

The following sample calculations are only exemplary theoretical calculations that are meant to provide one of ordinary skill in the art with calculations on the energy input and output of the system 100.

The amount of electrical input to the plasma torch=800 kilowatt hour per ton of municipal solid waste (2,720,000 British thermal units per ton of municipal solid waste). The syngas volume is 40,000 standard cubic feet per ton of municipal solid waste gasified. The exit temperature of the plasma converter is 2,100° F.

The thermal energy in gases produced by the plasma gasifier is given by the following equation (V):

=weight×sp. heat×Δt (V)

where the weight is 40,000 standard cubic feet (SCF) per ton (SCF/ton) of municipal solid waste, and Δt is 1,700° F. Δt=2000° F. (gasifier output temperature)−300° F. (minimum useful temperature).

The thermal energy for various gases produced in the plasma gasifier using the equation (V) is as follows:

$$\begin{matrix} For hydrogen ( {H}_{2} ) = 0.5 \times 40 , 000 \times 3.406 \times 1 , 700 \\ = 648 , 500 B T U \text{/} ton M S W\end{matrix}$$

$$\begin{matrix} For carbon monoxide = 0.4 \times 40 , 000 \times 0.2426 \times 1 , 700 \\ = 501 , 500 B T U \text{/} ton M S W\end{matrix}$$

$$For air = 0.1 \times 40 , 000 \times 0.239 \times 1 , 700 = 130 , 000 B T U \text{/} ton M S W$$

$$The total thermal energy in the gases is 1 , 280 , 000 B T U \text{/} ton M S W . \\ Heat released from the combustion of gases$$

$$From {H}_{2} = 319 B T U \text{/} S C F = 319 \times 20 , 000$$

$$6 , 380 , 000 B T U \text{/} Ton M S W$$

$$From C O = 331 B T U \text{/} S C F = 331 \times 16 , 000 \\ = 5 , 296 , 000 B T U \text{/} Ton M S W$$

$$Total = 6380000 + 5296000 = 11 , 676 , 000 B T U \text{/} Ton M S W$$

### Heat Balance Check

Energy Input=Energy in Syngas From Plasma Gasifier

MSW+Power to Plasma Torch=Heat in Gases+Heat release from gases

(5000×2000)+2,720,000=1,280,000+11,676,000

12,720,000=12,956,000(within 2% difference)

$$Power needed for plasma gasifier torch = 800 Kw hr \text{/} Ton M S W . \\ Power available from all energy in syngas = 40 \% \times 12956000 \text{/} 3400 ( assuming a 40 \% efficiency ) = \\ 1524 Kw hr \text{/} Ton M S W .$$

As a result, energy of 724 Kwhr/Ton of municipal solid waste is available for consumption by other sources (e.g., lighting in society, locomotives, or the like).

Heat in Syngas from the Plasma Gasifier
Energy Available from High Temperature Plasma Gasifier Heat Recovery:

$$With 30 \% of useful energy recovered = 0.3 \times 1 , 280 , 000 B T U \text{/} ton M S W . \\ = 0.3 \times 1 , 280 , 000 \text{/} 3400 Kw Hr \text{/} Ton M S W$$

$$Plasma Gasifier Energy Recovery = 108 Kw Hr \text{/} Ton M S W$$

Energy Available from MSW Using Syngas Engine Exhaust Gas Heat Recovery

Power in syngas combustion=11,676,000 BTU/Ton MSW

At 40% overall system efficiency=0.4×11,676,000/3400

Energy available from syngas combustion=1,374 Kw Hr/Ton MSW

Energy in exhaust gas at 30% of total=0.3×11,676,000/3400

Energy available in exhaust gas=1,030 Kw Hr/Ton MSW

At 30% heat recovery efficiency=309 Kw Hr/Ton MSW

Electrical Power Available from MSW:

| | |
|---|---|
| Syngas Combustion | 1374 KwHr/Ton MSW |
| Plasma Gasifier Syngas Heat Recovery | 108 |
| Syngas Engine Exhaust Heat Recovery | 309 |
| Total Electricity Generation | 1,781 Kw Hr/Ton MSW |

Plasma gasifier output (From 1 Ton MSW)

H₂ from syngas=0.0056×20,000=112 lbs

CO from syngas=0.076×16,000=1216 lbs

Other air from syngas=0.08×4,000=320 lbs

Carbon throughout flow loop=520 lbs

Oxygen in carbon monoxide=695 lbs

Boiler combustion

CO+O=CO₂

Therefore the oxygen produced is 695 lbs; the carbon produced is 520 lbs and the carbon dioxide produced in 1910 lbs. The oxygen used for stoichiometric combustion is 695 lbs.

### Sample Calculations for the Algae Bioreactor

Here it is assumed that oil is approximately half carbohydrate and half hydrocarbon oil. Oil is approximately half the total weight of the algae fraction.

Oil CH₂O+CH₂═C₂H₄O; Mol. Wt.=44

Remainder CH₂O; Mol. Wt.=30
For every 100 lbs of algae there would therefore be 50 lbs of oil (1.14 lb-moles) and 50 lbs of carbohydrate.

Thus, the balanced chemical equation is

3.95CO₂+3.95H₂O→C_{3.95}H_{7.90}O_{2.81}+4.52O₂

For the equation above, the amount of CO₂ absorbed is 1910 lbs.
From the equation above, the amount of carbon dioxide, water and hydrogen may be calculated as follows:

$${H}_{2} O = \frac{1910 \times 71.1}{173.8} = 780 lbs input ;$$

$$C = \frac{1910 \times 12}{44} = 521.2 lbs input in C {O}_{2} ;$$

$${O}_{2} = \frac{1910 \times 144.64}{73.8} = 1590 lbs output ;$$

$$Algae = 1 , 100 lbs of output .$$

### Operation of Water Gas Shift Reactor (Sample Calculations for the Water Gas Shift Reactor)

In the water gas shift reactor, steam is used to convert carbon monoxide to carbon dioxide as shown below:

$$C O + {H}_{2} O = C {O}_{2} + {H}_{2}$$

$$C O = 1220 lbs$$

$${H}_{2} O = \frac{1220 \times 18}{28} = 845 lbs input ( approximately 850 lbs ) ;$$

$$C {O}_{2} = \frac{1220 \times 44}{28} = 1910 lbs output ;$$

$${H}_{2} = \frac{1220 \times 2}{28} = 87 lbs output ;$$

where 18 is the molecular weight of water; 44 is the molecular weight of carbon dioxide and 2 is the molecular weight of hydrogen.

Total hydrogen produced in the water gas shift reactor=87+112=199 lbs output (approximately 200 lbs); where the 112 lbs of hydrogen are produced in the plasma gasifier as shown above.

### Sample Calculations for the Boiler, Fuel Cell or the Hydrogen Engine

The boiler, fuel cell and the hydrogen engine, all combust hydrogen and produce water. As noted above, the total hydrogen output in the water gas shift reactor=200 lbs; the corresponding amount of oxygen used to produce water is 200×8=1600 lbs. Thus the total amount of water (which is the sum of the hydrogen and oxygen is 1800 lbs.

### Sample Calculations for the Methanation Plant

For 1 ton of MSW, the water gas shift reaction can be described as follows: CO+H₂O═CO₂+H₂; where the heat of reaction ΔH_{25° C.}=9.85 Kcal/mole

The carbon monoxide methanation can be described as follows: CO+3H₂═CH₄+H₂O; where the heat of reaction ΔH_{25° C.}=−49.3 Kcal/mole

The carbon dioxide methanation can be described as follows: CO₂+4H₂═CH₄+2H₂O; where the heat of reaction ΔH_{25° C.}=3.94 Kcal/mole

This is the approximate amount found in one ton of MSW.

Calculating the amount of carbon monoxide, methane and water based on the weight of hydrogen being 112 lbs.

$$C O = \frac{112 \times 28}{6} = 523 lbs input ;$$

$$C {H}_{4} = \frac{112 \times 16}{6} = 298.7 lbs output$$

$${H}_{2} O = \frac{112 \times 18}{6} = 336 lbs output ;$$

where 28 is the molecular weight of carbon monoxide; 16 is the molecular weight of methane and 6 is the total weight of the hydrogen on the left hand side of the reaction for the carbon monoxide methanation above (where the heat of reaction ΔH_{25° C.}=−49.3 Kcal/mole).

The carbon monoxide remaining=1220−523=697 lbs; this is fed to the water gas shift reactor. The carbon dioxide, water and hydrogen produced in the water gas shift reactor are as follows (based on the 697 lbs of carbon monoxide)

$$C {O}_{2} = \frac{697 \times 44}{28} = 1095 lbs output$$

$${H}_{2} O = \frac{697 \times 18}{28} = 448 lbs input$$

$${H}_{2} = \frac{697 \times 2}{28} = 49.8 lbs input$$

where 28 is the molecular weight of carbon monoxide; 44 is the molecular weight of carbon dioxide; 18 is the molecular weight of water and 2 is the molecular weight of hydrogen.

In the methanation plant, the carbon dioxide methanation can be described as follows:

$$C {O}_{2} + 4 {H}_{2} = C {H}_{4} + 2 {H}_{2} O ;$$

$$where the heat of reaction Δ {H}_{25 ° C .} = 39.4 Kcal \text{/} mole$$

$$The amount of hydrogen produced = 49.8 lbs$$

$$The C {O}_{2} used = \frac{49.8 \times 44}{8} = 274 lbs$$

$$The C {O}_{2} remaining 1095 - 274 = 821 lbs$$

$$C {H}_{4} = \frac{49.8 \times 16}{8} = 99.6 lbs output$$

$${H}_{2} O = \frac{49.8 \times 36}{8} = 224 lbs output$$

The input and the output to the methanation system may be summed up as follows:

### INPUT

CO=1220lbs

H=112 lbs

### OUTPUT

CH₄=298.7+99.6=398.3lbs

CO₂=1095−274=821lbs

H₂O=(336+224)−448=112lbs

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention.

What is claimed is:

1. A system comprising:
a high temperature gasifier; the high temperature gasifier being operative to function at temperatures of greater than or equal to about 450° F.; and
a synthesizing reactor in fluid communication with the high temperature gasifier; the synthesizing reactor being operative to convert carbon dioxide into another form of carbon.

2. The system of claim 1, wherein the synthesizing reactor is an algae bioreactor, a photo plankton reactor, an enzyme reactor, a bacterial reactor or a combination comprising at least one of the foregoing synthesizing reactors.

3. The system of claim 1, wherein the synthesizing reactor is an algae bioreactor.

4. The system of claim 1, wherein the high temperature gasifier is a plasma gasifier.

5. The system of claim 4, wherein the plasma gasifiers heats up feedstock or liquidizes metal to transfer heat to the feedstock.

6. The system of claim 1, wherein the high temperature gasifier uses water to provide oxygen and hydrogen.

7. The system of claim 1, wherein the high temperature gasifier operates at a temperature of about 4000° F. to about 50,000° F.

8. The system of claim 1, wherein the high temperature gasifier is effective to convert carbonaceous materials into carbon monoxide and hydrogen; the carbonaceous materials comprising hydrocarbons, carbohydrates, coal, municipal solid waste from landfills, industrial waste streams or combinations thereof.

9. The system of claim 1, further comprising a carbon oxygenation reactor; the carbon oxygenation reactor being in fluid communication with the high temperature gasifier; the carbon oxygenation reactor being a syngas engine, a syngas engine electric generator, a syngas boiler, a syngas boiler electric generator, a water gas shift reactor, a catalytic converter, a methanation plant, or a combination comprising at least one of the foregoing.

10. The system of claim 9, wherein the system comprises a carbon loop; the carbon loop comprising the high temperature gasifier, the carbon oxygenation reactor and the synthesizing reactor.

11. The system of claim 10, wherein the carbon loop is an open loop or a closed loop.

12. The system of claim 10, wherein an amount of carbon circulating in the carbon loop is substantially constant.

13. The system of claim 1, wherein the system further comprises a hydrogen loop; the hydrogen loop comprising a carbon oxygenation reactor and a hydrogen oxygenation reactor; the hydrogen oxygenation reactor being a hydrogen engine, a hydrogen engine electric generator, a hydrogen boiler, a hydrogen boiler electric generator, a fuel cell, or a combination comprising at least one of the foregoing hydrogen oxygenation reactors.

14. The system of claim 1, wherein the synthesizing reactor is located down stream of a carbon oxygenation reactor and is operative to produce an oil rich carbohydrate.

15. The system of claim 9, wherein the carbon oxygenation reactor operates at temperatures of about 1800° F. to about 2200° F. to produce carbon dioxide.

16. The system of claim 1, further comprising a hydrogen separator that is located downstream of the high temperature gasifier.

17. The system of claim 1, further comprising a carbon oxygenation reactor that is located downstream of the high temperature gasifier and upstream of a hydrogen oxygenation reactor.

18. The system of claim 13, wherein the hydrogen loop is a closed loop and generates steam or electric power.

19. The system of claim 10, wherein no carbon dioxide is released out of the carbon loop.

20. The system of claim 1, wherein oil rich algae produced in the synthesizing reactor is gasified in the high temperature gasifier and wherein an average amount of carbon entering the carbon loop is equal to an average amount of carbon leaving the carbon loop.

21. The system of claim 1, wherein the synthesizing reactor utilizes artificial lighting.

22. The system of claim 10, wherein the carbon loop comprises carbon, oxygen or hydrogen in various forms.

23. The system of claim 10, wherein the carbon loop is used for harvesting hydrogen and oxygen.

24. The system of claim 1, wherein the synthesizing reactor is a source of oxygen for an energy generating device.

25. A power generation plant that uses the system of claim 1.

26. A method comprising:
gasifying a waste stream to produce a composition that comprises syngas; the syngas comprising carbon monoxide and hydrogen; the gasifying being conducted in a high temperature gasifier; the high temperature gasifier being operative to function at temperatures of greater than or equal to about 450° F.;
converting the carbon monoxide into carbon dioxide; and
feeding the carbon dioxide to an synthesizing reactor to produce an oil rich algae.

27. The method of claim 26, further comprising recycling the oil rich algae to the high temperature gasifier to undergo gasification.

28. The method of claim 26, wherein the carbon monoxide is converted to carbon dioxide in a carbon oxygenation reactor.

29. The method of claim 26, further comprising separating the carbon monoxide from the hydrogen.

30. The method of claim 26, further comprising igniting the syngas.

31. The method of claim 26, further comprising generating energy.
